(19) United States
(12) Patent Application Publication
(10) Pub. No.: US 2007/0042006 A1
(43) Pub. Date: Feb. 22, 2007

(54) Stable carprofen composition

(76) Inventor: Kai Kin Lau, New South Wales (AU)

Correspondence Address: SONNENSCHEIN NATH & ROSENTHAL LLP, P.O. BOX 061080, WACKER DRIVE STATION, SEARS TOWER, CHICAGO, IL, 60606-1080, US

(21) Appl. No.: 10/552,408

(22) PCT Filed: Dec. 24, 2003
(86) PCT No.: PCT/AU03/01729

§ 371 Date: Aug. 8, 2006

(30) Foreign Application Priority Data
Apr. 7, 2003 (AU) 2003/100262

(51) Int. Cl. A61K 31/403; A61K 9/00

(52) U.S. Cl. 424/400; 514/411

(57) ABSTRACT

A stable solvent-based composition is described which is particularly useful in warm blooded animals such as dogs. The composition comprises a therapeutically effective amount of carprofen, one or more polyols, one or more stabilising agents and optionally, one or more co-solvents.

### TECHNICAL FIELD

This invention relates to non-steroidal anti-inflammatory drug (NSAID) compositions and in particular to such compositions where the NSAID is presented in the form of a solution for use in warm blooded animals, such as dogs.

### BACKGROUND ART

There are a number of NSAID's that are known to be useful for the treatment of inflammation and pain in animals such as dogs. These NSAID's are typically used in treating postoperative pain associated with soft tissue and orthopaedic surgeries as well as for the relief of pain and inflammation associated with osteoarhritis.

One such useful NSAID is carprofen. This drug is a member of the class of drugs that includes indomethacin, naproxen and ketoprofen. Chemically, carprofen is 6-chloro-α-methyl-9H-carbazole-2-acetic acid.

Whilst carprofen has been found to be very effective therapeutically, in order to maintain an acceptable stability profile, it must be formulated in dosage forms such as tablets where solvents are largely excluded. For administration to humans, such dosage forms do not present a barrier to use. However, for administration to non-human animals, solid dosage forms are not well tolerated and are generally difficult to administer.

It would therefore be desirable if carprofen could be presented in a non-solid dosage form thereby allowing the substance to be more easily administered.

The present inventors have recognised this limitation on the use of carprofen and accordingly have sought to provide compositions that are stable and solvent-based for ease of administration to warm-blooded animals, especially dogs.

In the disclosure that follows, any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

Moreover, throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### SUMMARY OF THE INVENTION

The present inventors have achieved stable solvent-based compositions of carprofen through the finding that certain solvent combinations with carprofen result in formulations that are stable and are suitable for oral administration to animals.

Accordingly, in a first aspect, the present invention is directed to a stable solvent-based composition comprising:
a therapeutically effective amount of carprofen;
one or more polyols;
one or more stabilising agents; and optionally,
one or more co-solvents.

In a second aspect, the present invention is further directed to a method of treating pain and/or inflammation in a warm-blooded non-human animal, the method comprising administering to the animal a therapeutically effective amount of carprofen which is solubilised in a composition which comprises:
one or more polyols;
one or more stabilising agents; and optionally,
one or more co-solvents.

In a third aspect, the present invention is further directed to the use of a composition which comprises:
one or more polyols;
one or more stabilising agents; and optionally,
one or more co-solvents,
to stabilise carprofen and to facilitate the oral administration of a therapeutically effective amount of carprofen to a warm-blooded non-human animal.

In a fourth aspect, the present invention is still farther directed to use of a therapeutically effective amount of carprofen which is solubilised in a composition which comprises:
one or more polyols;
one or more stabilising agents; and optionally,
one or more co-solvents.
in the preparation of a medicament for treating pain and/or inflammation in a warm-blooded non-human animal.

Preferably, carprofen is included in the composition in an amount of about 1 to 500 g/L, more preferably about 5 to 50 g/L, even more preferably about 20 to 50 g/L. At these concentrations, an appropriately therapeutically effective amount of the composition may be administered to an animal.

One or more polyols are included in the composition and these may be selected from the group consisting of propylene glycol, glycerol, sorbitol, solid polyethylene glycols, liquid polyethylene glycols and mixtures of the foregoing. Broadly the polyols may be incorporated in an amount of from about 20 to 998 g/L. Preferably they are used in an amount of from about 700 to 998 g/L. In the case of sorbitol, it is usual to provide the sorbitol as a 70% w/v aqueous solution. In addition, in order for the polyethylene glycols to be liquid, there molecular weight will generally be in the range of about 300-600. However, potentially solid polyethylene glycols could be used in combination with one or more suitable co-solvents.

Amongst the stabilising agents that may be used are antioxidants. These include a tocopherol and salts thereof, ascorbic acid and salts thereof, methoxyphenol and derivatives thereof, trihydroxybenzoate and derivatives thereof, hydroquinone and derivatives thereof, methyl phenol and derivatives thereof and sodium metabisulfite. Generally these stabilising agents are regarded as antioxidants. In addition, benzyl alcohol may be used as a stabilising agent. Such stabilising agents may be used singly or in combination in a total amount of about 0.1 to 50 g/L, preferably about 10 to 20 g/L.

Optionally, one or more co-solvents may be included in the compositions of the invention. One co-solvent that may be used is ethanol. If a co-solvent is used, the amount is typically up to about 500 g/L, preferably about 10 to 300 g/L.

Although the compositions of the invention are solutions of carprofen, it will be readily appreciated that the viscosity of such solutions may be modified to produce compositions that are enhanced so as to be, for example, more paste like or in the form of a gel.

To produce the compositions of the invention, the carprofen may be dissolved in polyol along with the stabilising agent. If a co-solvent is used, it may be added following the dissolution of the carprofen and stabilising agent.

The compositions according to the present invention are for oral administration to warm-blooded animals, particularly dogs. For successful administration, these compositions must be palatable to the animal to be treated.

In a preferred embodiment according to the first aspect of the invention, there is provided a stable solvent-based composition comprising:

a therapeutically effective amount of carprofen in an amount of about 1 to 500 g/L;

one or more polyols in an amount of from about 20 to 998 g/L;

one or more stabilising agents in an amount of from about 0.1 to 50 g/L; and

one or more co-solvents in an amount of from about 0 to 500 g/L.

In an even more preferred embodiment according to the first aspect of the invention, there is provided a stable solvent-based composition comprising:

a therapeutically effective amount of carprofen in an amount of about 1 to 500 g/L;

one or more polyols in an amount of from about 20 to 998 g/L, wherein the one or more polyols are selected from the group consisting of propylene glycol, glycerol, sorbitol, solid polyethylene glycols and liquid polyethylene glycols and mixtures of the foregoing;

one or more stabilising agents in an amount of from about 0.1 to 50 g/L, wherein the one or more stabilising agents are selected from the group consisting of a tocopherol and salts thereof, ascorbic acid and salts thereof, methoxyphenol and derivatives thereof, trihydroxybenzoate and derivatives thereof, hydroquinone and derivatives thereof, methyl phenol and derivatives thereof, sodium metabisulfite and benzyl alcohol; and

a co-solvents in an amount of from about 0 to 500 g/L, wherein the co-solvent is ethanol.

In a preferred embodiment of the second aspect of the invention, there is provided a method of treating pain and/or inflammation in a warm-blooded non-human animal, the method comprising administering to the animal a therapeutically effective amount of carprofen in an amount of about 1 to 500 g/L which is solubilised in a composition which comprises:

one or more polyols in an amount of from about 20 to 998 g/L, wherein the one or more polyols are selected from the group consisting of propylene glycol, glycerol, sorbitol, solid polyethylene glycols and liquid polyethylene glycols and mixtures of the foregoing;

one or more stabilising agents in an amount of from about 0.1 to 50 g/L, wherein the one or more stabilising agents are selected from the group consisting of a tocopherol and salts thereof, ascorbic acid and salts thereof, methoxyphenol and derivatives thereof, trihydroxybenzoate and derivatives thereof, hydroquinone and derivatives thereof, methyl phenol and derivatives thereof, sodium metabisulfite and benzyl alcohol; and

a co-solvents in an amount of from about 0 to 500 g/L, wherein the co-solvent is ethanol.

In a preferred embodiment of the third aspect of the invention, there is provided use of a composition which comprises:

one or more polyols in an amount of from about 20 to 998 g/L, wherein the one or more polyols are selected from the group consisting of propylene glycol, glycerol, sorbitol, solid polyethylene glycols and liquid polyethylene glycols and mixtures of the foregoing;

one or more stabilising agents in an amount of from about 0.1 to 50 g/L, wherein the one or more stabilising agents are selected from the group consisting of a tocopherol and salts thereof, ascorbic acid and salts thereof, methoxyphenol and derivatives thereof, trihydroxybenzoate and derivatives thereof, hydroquinone and derivatives thereof, methyl phenol and derivatives thereof, sodium metabisulfite and benzyl alcohol; and optionally,

a co-solvents in an amount of from about 0 to 500 g/L, wherein the co-solvent is ethanol;

to stabilise carprofen and to facilitate the oral administration of a therapeutically effective amount of carprofen in an amount of about 1 to 500 g/L to a warm-blooded non-human animal.

In a preferred embodiment of the fourth aspect of the invention, there is provided use of a therapeutically effective amount of carprofen in an amount of about 1 to 500 g/ which is solubilised in a composition which comprises:

one or more polyols in an amount of from about 20 to 998 g/L, wherein the one or more polyols are selected from the group consisting of propylene glycol, glycerol, sorbitol, solid polyethylene glycols and liquid polyethylene glycols and mixtures of the foregoing;

one or more stabilising agents in an amount of from about 0.1 to 50 g/L, wherein the one or more stabilising agents are selected from the group consisting of a tocopherol and salts thereof, ascorbic acid and salts thereof, methoxyphenol and derivatives thereof, trihydroxybenzoate and derivatives thereof, hydroquinone and derivatives thereof, methyl phenol and derivatives thereof, sodium metabisulfite and benzyl alcohol; and optionally,

a co-solvents in an amount of from about 0 to 500 g/L, wherein the co-solvent is ethanol;

in the preparation of a medicament for treating pain and/or inflammation in a warm-blooded non-human animal.

### BRIEF DESCRIPTION OF THE FIGURES

FIG. 1 is a comparison of the average carprofen concentration versus time profile between a stable liquid composition according to the present invention and Rimadyl® tablets.

### MODES FOR CARRYING OUT THE INVENTION

In order to better understand the nature of this invention, a number of examples will now be described.

### EXAMPLE 1

| Ingredient | Amount | |
|---|---|---|
| Carprofen | 25 | g |
| Butylated hydroxytoluene | 1 | g |
| Ethanol | 100 | mL |
| Polyethylene glycol 400 | qs 500 | mL |

### EXAMPLE 2

| Ingredient | Amount | |
|---|---|---|
| Carprofen | 10 | g |
| Butylated hydroxyanisole | 2 | g |
| Sorbitol 70% aqueous solution | qs 500 | mL |

### EXAMPLE 3

| Ingredient | Amount | |
|---|---|---|
| Carprofen | 10 | g |
| Butylated hydroxytoluene | 1 | g |
| Sorbitol 70% aqueous solution | 100 | mL |
| Propylene glycol | qs 500 | mL |

### EXAMPLE 4

| Ingredient | Amount | |
|---|---|---|
| Carprofen | 25 | g |
| Butylated hydroxyanisole | 2 | g |
| Polyethylene glycol 400 | 400 | mL |
| Ascorbic acid | 5 | g |
| Ethanol | qs 500 | mL |

### EXAMPLE 5

| Ingredient | Amount | |
|---|---|---|
| Carprofen | 20 | g |
| Propylene glycol | qs to 1 | L |
| Benzyl alcohol | 10 | g |

### EXAMPLE 6

| Ingredient | Amount | |
|---|---|---|
| Carprofen | 20 | g |
| Butylated hydroxytoluene | 5 | g |
| Ethylene glycol | qs to 1 | L |

### EXAMPLE 7

| Ingredient | Amount | |
|---|---|---|
| Carprofen | 20 | g |
| Benzyl alcohol | 10 | g |
| Butylated hydroxytoluene | 2 | g |
| Propylene glycol | qs to 1 | L |

In Examples 1-7, each composition was prepared by dissolving the carprofen in the polyol. The stabilising agent was then dissolved and if appropriate, co-solvent was added to complete the formulations. The availability of all of the ingredients used in Examples 1-7 is set out in Table 1.

Stability Study

The stability of the compositions described in Examples 3, 6 and 7 was evaluated by storing samples for various times at 30 and 40° C. The results of these stability trials are set out in Tables 2-4 from which it can be seen that the samples were stable for the time tested. By comparison, an example tested that did not incorporate a stabilising agent, had degraded to an unacceptable level of carprofen after 1-3 months storage at 30° C.

### EXAMPLE 8

### Bioequivalent Study

A bioequivalence study in dogs of carprofen formulated as a liquid composition according to Example 7 (containing 20 mg carprofen/mL) to Rimadyl® tablets (20 mg per tablet; Pfizer Animal Health) after oral administration at 4 mg/kg was evaluated by the pharmacokinetic parameters area under the plasma concentration-time curve to infinity (AUC_{0-inf}), and maximum drug concentration (Cₘₐₓ).

Study Design

Twelve healthy adult dogs (6 Male, 6 Female) were orally dosed at 4 mg carprofen/kg body weight with each of the test and reference formulations in a randomised cross-over design with a 14 day washout period. Blood samples were drawn before and at prescribed intervals after dosing. Plasma was separated from the blood, then frozen and stored until it was analyzed for total racemate carprofen concentration by LCMSMS. Plasma concentration versus time data was analysed using bioequivalence comparison according to the method of Westlake as implemented in WinNonlin version 2.0 (Pharsight Corp, USA).

Study Results

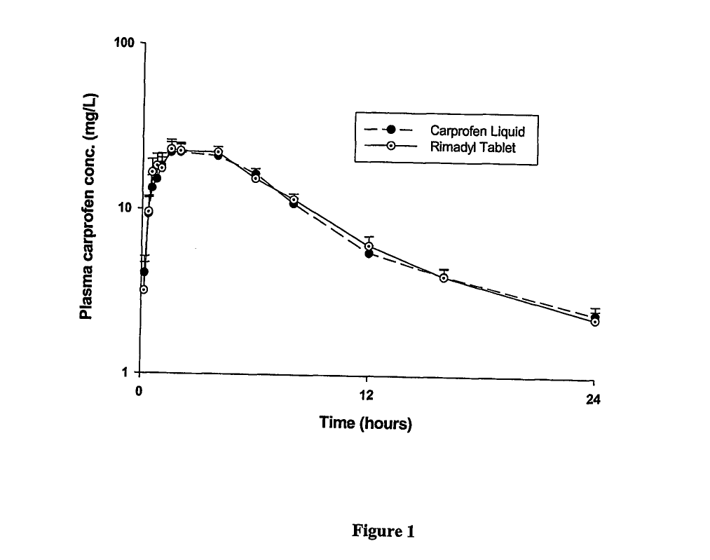

Plasma harvested from the blood samples was frozen prior to transport for carprofen analysis. Comparison of the average plasma carprofen concentration versus time profiles of Carprofen Liquid versus Rimadyl® tablets is shown if FIG. 1. Time to maximum concentration (Tmax), maximum concentration (Cmax), and area under the curve (AUC) were calculated for individual animals from the plasma carprofen concentrations and compared for the two formulations. With respect to AUC_{(0inf)}, the confidence interval for Carprofen Liquid was within 80-120% of the confidence interval for Rimadyl® Tablets, and therefore met the criteria for bioequivalence. The comparison for Cmax, however, fell outside the interval defined for bioequivalence, even though by ANOVA the effect of formulation on Cmax was not significant (p=0.5557). The power for the Cₘₐₓ comparison was low (0.34), and it is likely if more animals had been included in the study, bioequivalence as determined by Cₘₐₓ would have been demonstrated.

Carprofen Liquid administered orally to dogs at a dose rate of 4 mg carprofen per kg body weight was found to be bioequivalent to Rimadyl Tablets with respect to AUC_{(0-inf)} as indicated by plasma carprofen concentrations. The two formulations were not quite bioequivalent with respect to Cmax

TABLE 1
Ingredient Availability

| Ingredient | Available from |
|---|---|
| Carprofen | Pacific Resources |
| | International Pty Ltd |
| Butylated hydroxyanisole | Bronson & Jacobs |
| Polyethylene glycol 400 | Bronson & Jacobs |
| Ascorbic acid | Bronson & Jacobs |
| Ethanol | CSR |
| Butylated hydroxytoluene | Bronson & Jacobs |
| Sorbitol | Bronson & Jacobs |
| Propylene glycol | Bronson & Jacobs |
| Benzyl alcohol | Bronson & Jacobs |
| Ethylene glycol | Bronson & Jacobs |

TABLE 2
Stability Evaluation of Example 3

| Storage Time | Carprofen (g/L) | Carprofen (g/L) |
|---|---|---|
| (months) | Temperature 30° C. | Temperature 40° C. |
| Initial | 19.8 | 19.8 |
| 3 | 19.9 | 20.2 |
| 6 | 20.1 | 19.9 |
| 9 | 19.7 | 20.3 |

TABLE 3
Stability Evaluation of Example 6

| Storage Time | Carprofen (g/L) | Carprofen (g/L) |
|---|---|---|
| (months) | Temperature 30° C. | Temperature 40° C. |
| Initial | 21.0 | 21.0 |
| 3 | 21.0 | 21.0 |
| 6 | 20.6 | 20.6 |
| 12 | 20.0 | 19.8 |

TABLE 4
Stability Evaluation of Example 7

| Storage Time | Carprofen (g/L) | Carprofen (g/L) |
|---|---|---|
| (months) | Temperature 30° C. | Temperature 40° C. |
| Initial | 21.5 | 21.5 |
| 2 | 20.9 | 20.6 |
| 3 | 21.0 | 21.1 |
| 6 | 20.6 | 20.0 |
| 9 | 19.9 | 19.3 |
| 12 | 19.8 | 19.2 |
| 18 | 19.6 | Not tested |

1. A stable solution formulation consisting essentially of:
a therapeutically effective amount of carprofen;
one or more polyols in an amount of from about 20 to 998 g/L;
one or more stabilising agents in an amount of from about 0.1 to 50 g/L; and
one or more co-solvents in an amount of from about 0 to 500 g/L.

2. A stable solution composition consisting of:
a therapeutically effective amount of carprofen;
one or more polyols in an amount of from about 20 to 998 g/L;
one or more stabilising agents in an amount of from about 0.1 to 50 g/L; and
one or more co-solvents in an amount of from about 0 to 500 g/L.

3. The solution formulation according to claim 1 wherein the one or more polyols are selected from the group consisting of propylene glycol, glycerol, sorbitol, solid polyethylene glycols and liquid polyethylene glycols and mixtures of the foregoing; the one or more stabilising agents are selected from the group consisting of a tocopherol and salts thereof, ascorbic acid and salts thereof, methoxyphenol and derivatives thereof, trihydroxybenzoate and derivatives thereof, hydroquinone and derivatives thereof, methyl phenol and derivatives thereof, sodium metabisulfite and benzyl alcohol.

4. The solution formulation according to claim 1, wherein the carprofen is in an amount of from about 1 to 500 g/L.

5. The solution formulation according to claim 1 wherein the carprofen is in an amount of from about 5 to 50 g/L.

6. The solution formulation according to claim 1 wherein the one or more polyols are in an amount of from about 700 to 998 g/L.

7. The solution formulation according to claim 1 wherein the one or more stabilising agents are in an amount of from about 10 to 20 g/L.

8. The solution formulation according to claim 1 wherein the one or more co-solvents are in an amount of from about 10 to 300 g/L.

9. Use of a mixture consisting essentially of:
one or more polyols:
one or more stabilising agents; and optionally,
one or more co-solvents,
to solubilise and stabilise carprofen and to facilitate the oral administration of a therapeutically effective amount of carprofen to a warm-blooded non-human animal.

10. Use of a composition consisting of:
one or more polyols;
one or more stabilising agents; and optionally,
one or more co-solvents,
to solubilise and stabilise carprofen and to facilitate the oral administration of a therapeutically effective amount of carprofen to a warm-blooded non-human animal.

11. Use of a therapeutically effective amount of carprofen which is solubilised in a mixture which consists essentially of:
one or more polyols;
one or more stabilising agents; and optionally,
one or more co-solvents,
in the preparation of a medicament for treating pain and/or inflammation in a warm-blooded non-human animal.

12. Use of a therapeutically effective amount of carprofen which is solubilised in a composition which consist of:
one or more polyols;
one or more stabilising agents; and optionally,
one or more co-solvents,
in the preparation of a medicament for treating pain and/or inflammation in a warm-blooded non-human animal.

13. The use according to claim 9 wherein the one or more polyols are selected from the group consisting of propylene glycol, glycerol, sorbitol, solid polyethylene glycols and liquid polyethylene glycols and mixtures of the foregoing; the one or more stabilising agents are selected from the group consisting of a tocopherol and salts thereof, ascorbic acid and salts thereof, methoxyphenol and derivatives thereof, trihydroxybenzoate and derivatives thereof, hydroquinone and derivatives thereof, methyl phenol and derivatives thereof, sodium metabisulfite and benzyl alcohol.

14. The use according to claim 9 wherein the carprofen is in an amount of from about 1 to 500 g/L.

15. The use according to claim 9 wherein the carprofen is in an amount of from about 20 to 50 g/L.

16. The use according to claim 9 wherein the one or more polyols are in an amount of from about 700 to 998 g/L.

17. The use according to claim 9 wherein the one or more stabilising agents are in an amount of from about 10 to 20 g/L.

18. The use according to claim 9 wherein the one or more co-solvents are in an amount of from about 10 to 300 g/L.

19 A method of treating pain and/or inflammation in a warm-blooded non-human animal, the method comprising administering to the animal a solution formulation as defined in claim 1.

20. The method of claim 19 wherein the composition is administered orally.

21. (canceled)

22. (canceled)

23. The use according to claim 11 wherein the one or more polyols are selected from the group consisting of propylene glycol, glycerol, sorbitol, solid polyethylene glycols and liquid polyethylene glycols and mixtures of the foregoing; the one or more stabilising agents are selected from the group consisting of a tocopherol and salts thereof, ascorbic acid and salts thereof, methoxyphenol and derivatives thereof, trihydroxybenzoate and derivatives thereof, hydroquinone and derivatives thereof, methyl phenol and derivatives thereof, sodium metabisulfite and benzyl alcohol.

24. The use according to claim 11 wherein the carprofen is in an amount of from about 1 to 500 g/L.

25. The use according to claim 11 wherein the carprofen is in an amount of from about 20 to 50 g/L.

26. The use according to claim 11 wherein one or more polyols are in an amount of from about 700 to 998 g/L.
